(19) United States
(12) Patent Application Publication
(10) Pub. No.: US 2007/0134263 A1
(43) Pub. Date: Jun. 14, 2007

(54) Polypeptides for inducing a protective immune response against staphyloococcus aureus

(76) Inventors: Annaliesa S. Anderson, Doylestown, PA (US); Donna L. Montgomery, Chalfont, PA (US)

Correspondence Address: MERCK AND CO., INC, P O BOX 2000, RAHWAY, NJ, 07065-0907, US

(21) Appl. No.: 10/589,381

(22) PCT Filed: Feb. 14, 2005
(86) PCT No.: PCT/US05/04431

§ 371 Date: Aug. 15, 2006

Related U.S. Application Data
(60) Provisional application No. 60/545,447, filed on Feb. 18, 2004.

(51) Int. Cl. A61K 39/02; A61K 39/085; C07H 21/04; C12P 21/06; C12N 15/74; C12N 1/21; C07K 14/31

(52) U.S. Cl. 424/190.1; 424/243.1; 435/69.1; 435/252.3; 435/471; 530/350; 536/23.7

(57) ABSTRACT

The present invention features polypeptides comprising an amino acid sequence structurally related to SEQ ID NO: 1 and uses of such polypeptides. SEQ ID NO: 1 is a truncated derivative of a full length S. aureus polypeptide. The full-length polypeptide is referred to herein as full-length “sai-1”. A His-tagged derivative of SEQ ID NO: 1 was found to produce a protective immune response against S. aureus.

### CROSS-REFERENCE TO RELATED APPLICATIONS

The present application claims the benefit of U.S. Provisional Application No. 60/545,447, filed Feb. 18, 2004, which is hereby incorporated by reference herein.

### BACKGROUND OF THE INVENTION

The references cited throughout the present application are not admitted to be prior art to the claimed invention.

Staphylococcus aureus is a pathogen responsible for a wide range of diseases and conditions. Examples of diseases and conditions caused by S. aureus include bacteremia, infective endocarditis, folliculitis, furuncle, carbuncle, impetigo, bullous impetigo, cellulitis, botryomyosis, toxic shock syndrome, scalded skin syndrome, central nervous system infections, infective and inflammatory eye disease, osteomyletitis and other infections of joints and bones, and respiratory tract infections. (The Staphylococci in Human Disease, Crossley and Archer (eds.), Churchill Livingstone Inc. 1997.)

Immunological based strategies can be employed to control S. aureus infections and the spread of S. aureus. Immunological based strategies include passive and active immunization. Passive immunization employs immunoglobulins targeting S. aureus. Active immunization induces immune responses against S. aureus.

Potential S. aureus vaccines target S. aureus polysaccharides and polypeptides. Targeting can be achieved using suitable S. aureus polysaccharides or polypeptides as vaccine components. Examples of polysaccharides that may be employed as possible vaccine components include S. aureus type 5 and type 8 capsular polysaccharides. (Shinefield et al., N. Eng. J. Med. 346:491-496, 2002.) Examples of polypeptides that may be employed as possible vaccine components include collagen adhesin, fibrinogen binding proteins, and clumping factor. (Mamo et al., FEMS Immunology and Medical Microbiology 10:47-54, 1994, Nilsson et al., J. Clin. Invest. 101:2640-2649, 1998, Josefsson et al., The Journal of Infectious Diseases 184:1572-1580, 2001.)

Information concerning S. aureus polypeptide sequences has been obtained from sequencing the S. aureus genome. (Kuroda et al., Lancet 357:1225-1240, 2001, Baba et al., Lancet 359:1819-1827, 2000, Kunsch et al., European Patent Publication EP 0 786 519, published Jul. 30, 1997.) To some extent bioinformatics has been employed in efforts to characterize polypeptide sequences obtained from genome sequencing. (Kunsch et al., European Patent Publication EP 0 786 519, published Jul. 30, 1997.)

Techniques such as those involving display technology and sera from infected patients have been used in an effort to identify genes coding for potential antigens. (Foster et al., International Publication Number WO 01/98499, published Dec. 27, 2001, Meinke et al., International Publication Number WO 02/059148, published Aug. 1, 2002, Etz et al., PNAS 99:6573-6578, 2002.)

### SUMMARY OF THE INVENTION

The present invention features polypeptides comprising an amino acid sequence structurally related to SEQ ID NO: 1 and uses of such polypeptides. SEQ ID NO: 1 is a truncated derivative of a full length S. aureus polypeptide. The full-length polypeptide is referred to herein as full length “sai-1”. A His-tagged derivative of SEQ ID NO: 1 was found to produce a protective immune response against S. aureus.

Reference to “protective” immunity or immune response indicates a detectable level of protection against S. aureus infection. The level of protection can be assessed using animal models such as those described herein.

Thus, a first aspect of the present invention describes a polypeptide immunogen comprising an amino acid sequence at least 85% identical to SEQ ID NO: 1, wherein the polypeptide does not contain a carboxyl terminus provided by amino acids 261-294 of SEQ ID NO: 7 and the polypeptide provides protective immunity against S. aureus. SEQ ID NO: 7 provides a full length sai-1 polypeptide, wherein amino acids 261-294 provide the carboxyl terminus domain starting at the LPXTG motif.

Reference to “immunogen” indicates the ability to produce a protective immune response.

Reference to comprising an amino acid sequence at least 85% identical to SEQ ID NO: 1 indicates that a SEQ ID NO: 1 related region is present and additional polypeptide regions may be present. If additional polypeptide regions are present, then the polypeptide does not have a carboxyl LPXTG motif as provided by amino acids 261-294 of SEQ ID NO: 7.

Percent identity (also referred to as percent identical) to a reference sequence is determined by aligning the polypeptide sequence with the reference sequence and determining the number of identical amino acids in the corresponding regions. This number is divided by the total number of amino acids in the reference sequence (e.g., SEQ ID NO: 1) and then multiplied by 100 and rounded to the nearest whole number.

Another aspect of the present invention describes an immunogen comprising a polypeptide that provides protective immunity against S. aureus. The immunogen consists of the polypeptide and one or more additional regions or moieties covalently joined to the polypeptide at the carboxyl terminus or amino terminus, wherein each region or moiety is independently selected from a region or moiety having at least one of the following properties: enhances the immune response, facilitates purification, or facilitates polypeptide stability.

Reference to “additional region or moiety” indicates a region or moiety different from a sai-1 region. The additional region or moiety can be, for example, an additional polypeptide region or a non-peptide region.

Another aspect of the present invention describes a composition able to induce protective immunity against S. aureus in a patient. The composition comprises a pharmaceutically acceptable carrier and an immunologically effective amount of an immunogen that provides protective immunity against S. aureus.

An immunologically effective amount is an amount sufficient to provide protective immunity against S. aureus infection. The amount should be sufficient to significantly prevent the likelihood or severity of a S. aureus infection.

Another aspect of the present invention describes a nucleic acid comprising a recombinant gene encoding a polypeptide that provides protective immunity against S. aureus. A recombinant gene contains recombinant nucleic acid encoding a polypeptide along with regulatory elements for proper transcription and processing (which may include translational and post translational elements). The recombinant gene can exist independent of a host genome or can be part of a host-genome.

A recombinant nucleic acid is nucleic acid that by virtue of its sequence and/or form does not occur in nature. Examples of recombinant nucleic acid include purified nucleic acid, two or more nucleic acid regions combined together that provides a different nucleic acid than found in nature, and the absence of one or more nucleic acid regions (e.g., upstream or downstream regions) that are naturally associated with each other.

Another aspect of the present invention describes a recombinant cell. The cell comprises a recombinant gene encoding a polypeptide that provides protective immunity against S. aureus.

Another aspect of the present invention describes a method of making a polypeptide that provides protective immunity against S. aureus. The method involves growing a recombinant cell containing recombinant nucleic acid encoding the polypeptide and purifying the polypeptide.

Another aspect of the present invention describes a polypeptide that provides protective immunity against S. aureus made by a process comprising the steps of growing the recombinant cell containing recombinant nucleic acid encoding the polypeptide in a host and purifying the polypeptide. Different host cells can be employed.

Another aspect of the present invention describes a method of inducing a protective immune response in a patient against S. aureus. The method comprises the step of administering to the patient an immunologically effective amount of an immunogen that provides protective immunity against S. aureus.

Unless particular terms are mutually exclusive, reference to “or” indicates either or both possibilities. Occasionally phrases such as “and/or” are used to highlight either or both possibilities.

Reference to open-ended terms such as “comprises” allows for additional elements or steps. Occasionally phrases such as “one or more” are used with or without open-ended terms to highlight the possibility of additional elements or steps.

Unless explicitly stated reference to terms such as “a” or “an” is not limited to one. For example, “a cell” does not exclude “cells”. Occasionally phrases such as one or more are used to highlight the possible presence of a plurality.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 illustrates the amino acid sequence of SEQ ID NO: 1.

FIG. 2 illustrates the amino acid sequence of SEQ ID NO: 2.

FIG. 3 illustrate a sequence comparison between SEQ ID NO: 3 (SEQ 3), SEQ ID NO: 4 (SEQ 4), SEQ ID NO: 5 (SEQ 5), SEQ ID NO: 6 (SEQ 6), SEQ ID NO: 7 (SEQ 7), SEQ ID NO: 8 (SEQ 8), and SEQ ID NO: 9 (SEQ 9). SEQ ID NO: 3 is an amino His-tagged construct of SEQ ID NO: 1. SEQ ID NO: 4 is an amino His-tagged construct of SEQ ID NO: 2. SEQ ID NO: 5 is a carboxyl His-tagged construct of SEQ ID NO: 1. SEQ ID NO: 6 is an amino His-tagged construct of SEQ ID NO: 7. SEQ ID NO: 7 is full length COL sai-1 sequence. SEQ ID NO: 8 is sai-1 ATTC #ABO42826. SEQ ID NO: 9 is a carboxyl His-tagged construct of SEQ ID NO: 7.

FIG. 4 illustrates a nucleic acid sequence encoding SEQ ID NO: 3. The region encoding amino acids 3-260 of SEQ ID NO: 1 is shown in bold.

FIG. 5 illustrates a nucleic acid sequence encoding for SEQ ID NO: 4. The region encoding amino acids 3-264 of SEQ ID NO: 2 is shown in bold.

FIGS. 6A and 6B show an exemplary Coomassie stain of an SDS-PAGE gel and a Western blot, respectively, comparing intracellular expression from nucleic acid encoding SEQ ID NO: 1 related proteins. The Western blot was probed using an anti-his antibody. Lanes-1, Purified SEQ ID NO: 3 (100 ng); 2, SEQ ID NO: 3 E. coli crude lysate (with induction); 3, SEQ ID NO: 3 E. coli crude lysate (no induction); 4, SEQ ID NO: 5 E. coli crude lysate (with induction); 5, SEQ ID NO: 5 E. coli crude lysate (no induction); 6, SEQ ID NO: 6 E. coli crude lysate (with induction); 7, SEQ ID NO: 6 E. coli crude lysate (no induction); 8, SEQ ID NO: 9 E. coli crude lysate (with induction); 9, SEQ ID NO: 9 E. coli crude lysate (no induction); 10, Standard.

FIGS. 7A and 7B illustrate survival data from separate experiments using a SEQ ID NO: 3 polypeptide in aluminum hydroxyphosphate adjuvant (AHP). The polypeptide is referred to as “SEQ 3” is FIG. 7A and “Vaccine” in FIG. 7B.

### DETAILED DESCRIPTION OF THE INVENTION

SEQ ID NO: 1 is a truncated derivative of a S. aureus transferrin binding protein. An amino His-tagged derivative of SEQ ID NO: 1 was found to be expressed well in E. coli and to provide protective immunity against S. aureus infection. (See Example infra.) SEQ ID NO: 1 was produced based on a full length transferrin binding protein by modifying the encoding nucleic acid to remove the amino signal sequence, to remove a carboxyl hydrophobic region, to add an amino terminus methionine, and to add a restriction site to the amino terminus. The removed hydrophobic region followed a LPXTG motif. The addition of the amino terminus restriction site resulted in a Serine to Glycine substitution.

The ability of polypeptides structurally related to SEQ ID NO: 1 to provide protective immunity is illustrated using a polypeptide of SEQ ID NO: 3. SEQ ID NO: 3 is an amino His-tag derivative of SEQ ID NO: 1. The His-tag facilitates polypeptide purification and identification.

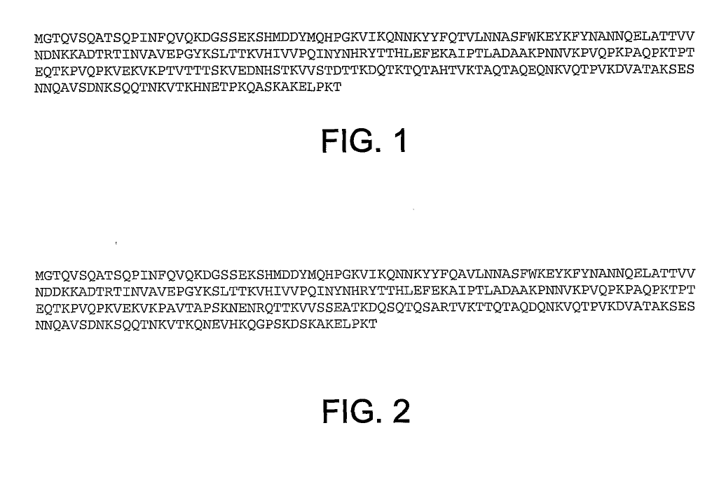

Polypeptides structurally related to SEQ ID NO: 1 include polypeptides containing corresponding regions present in different S. aureus strains and derivatives of naturally occurring regions. The amino acid sequence of SEQ ID NO: 1 is illustrated in FIG. 1. FIG. 2 (SEQ ID NO: 2) illustrates an example of a corresponding region found in a different S. aureus strain modified in a similar manner as SEQ ID NO: 1.

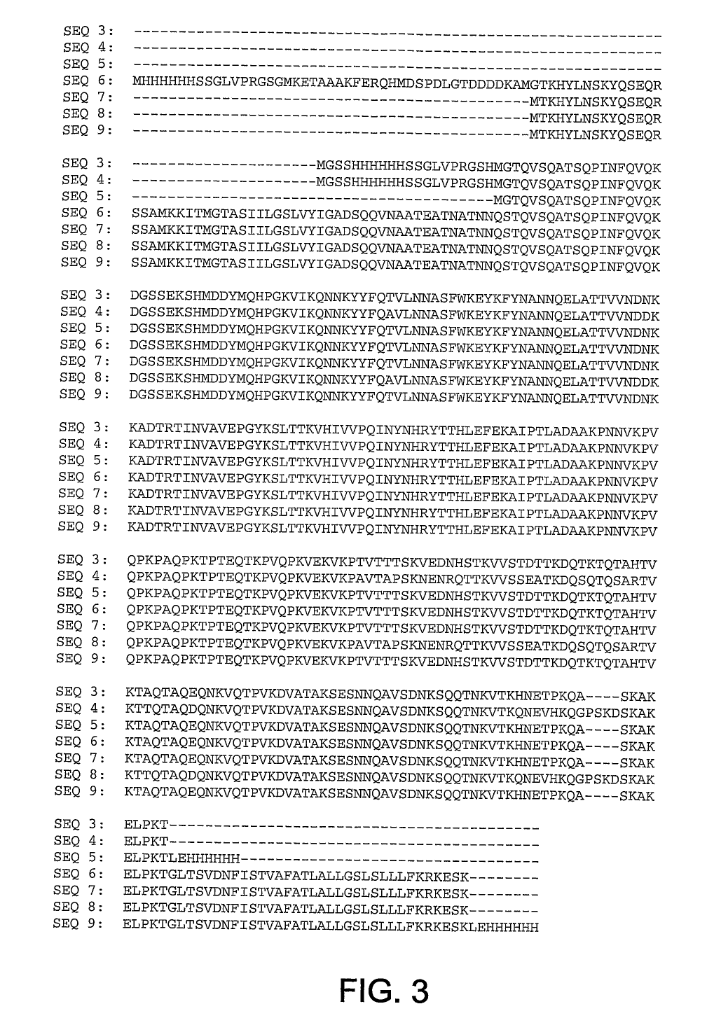

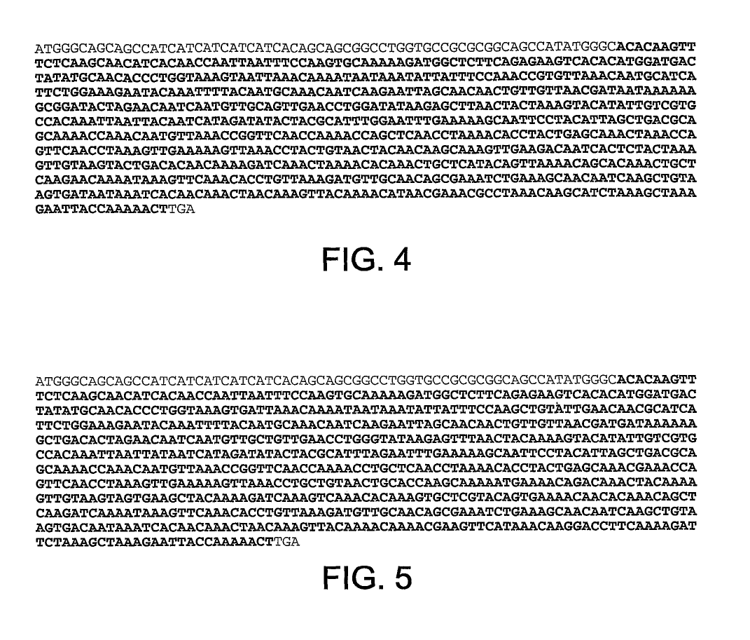

SEQ ID NO: 1 and SEQ ID NO: 2 are based on different naturally occurring full length S. aureus sai-1 sequences (SEQ ID NOs: 7 and 8). FIG. 3 provides a sequence comparison that includes SEQ ID NOs: 7 and 8 along with different His-tagged constructs based on SEQ ID NOs: 1, 2 and 7.

Sai-1 Sequences

Sai-1 sequences have been given different designations in different references. Examples of different designations are provided in Kuroda et al., Lancet 357:1225-1240, 2001 (SAV1130 and SA0977); Baba et al., Lancet 359:1819-1827, 2002 (MW1012); Mazmanian et al Molecular Microbiology 40(5):1049-1057, 2001 (SasE); Taylor and Heinrichs Mol. Microbiol. 43(6):1603-1614 (StbA), 2002; and Mazmanian et al., PNAS 99(4):2293-2298, 2002 and Mazmanian et al., Science 299:906-909, 2003 (IsdA).

A polypeptide sequence corresponding to a sai-1 protein sequence appears to be provided in different patent publications. (Meinke et al., International Publication Number WO 02/059148, published Aug. 1, 2002, Masignani et al., International Publication Number WO 02/094868, published Nov. 28, 2002, Foster et al., International Publication Number WO 02/102829, published Dec. 27, 2002, and Foster et al., International Publication Number WO 03/011899, published Feb. 13, 2003.)

Different sai-1 sequences may be present in different strains of S. aureus. Two examples of sai-1 sequences are provided by SEQ ID NO: 7 and 8. Other naturally occurring sai-1 sequences can be identified based on the presence of a high degree of sequence similarity or contiguous amino acids compared to a known sai-1 sequence. Contiguous amino acids provide characteristic tags. In different embodiments, a naturally occurring sai-1 sequence is a sequence found in a Staphylococcus, preferably S. aureus, having at least 20, at least 30, or at least 50 contiguous amino acids as in SEQ ID NO: 1; and/or having at least 85% sequence similarity or identity with SEQ ID NO: 1.

Sequence similarity can be determined by different algorithms and techniques well known in the art. Generally, sequence similarity is determined by techniques aligning two sequences to obtain maximum amino acid identity, allowing for gaps, additions and substitutions in one of the sequences.

Sequence similarity can be determined, for example, using a local alignment tool utilizing the program lalign (developed by Huang and Miller, Adv. Appl. Math. 12:337-357, 1991, for the <<sim>> program). The options and environment variables are:−f # Penalty for the first residue a gap (−14 by default); −g # Penalty for each additional residue in a gap (−4 by default)−s str (SMATRIX) the filename of an alternative scoring matrix file. For protein sequences, PAM250 is used by default−w # (LINLEN) output line length for sequence alignments (60).

SEQ ID NO: 1 Related Polypeptides

SEQ ID NO: 1 related polypeptides contain an amino acid sequence at least 85% identical to SEQ ID NO: 1. Reference to “polypeptide” does not provide a minimum or maximum size limitation.

A polypeptide at least 85% identical to SEQ ID NO: 1 contains up to 26 amino acid alterations from SEQ ID NO: 1. SEQ ID NO: 2 is an example of a polypeptide structurally related to SEQ ID NO: 1. In different embodiments, the SEQ ID NO: 1 related polypeptide is at 90%, least 94%, or at least 99% identical to SEQ ID NO: 1; at least 94% or 99% identical to SEQ ID NO: 2; differs from SEQ ID NO: 1 or SEQ ID NO: 2 by 0, 1, 2, 3, 4, 5, 6, 7, 8, 9, 10, 11, 12, 13, 14, 15, 16, 17, 18, 19, or 20 amino acid alterations; or consists essentially of amino acids 3-260 of SEQ ID NO: 1 or 3-264 of SEQ ID NO: 2. Each amino acid alteration is independently an addition, deletion or substitution.

Reference to “consists essentially” of indicated amino acids indicates that the referred to amino acids are present and additional amino acids may be present. The additional amino acids can be at the carboxyl or amino terminus. In different embodiments 1, 2, 3, 4, 5, 6, 7, 8, 9, 10, 11, 12, 13, 14, 15, 16, 17, 18, 19, or 20 additional amino acids are present. A preferred additional amino acid is an amino terminus methionine.

Alterations can be made to SEQ ID NOs: 1 or 2 to obtain derivatives that can induce protective immunity against S. aureus. Alterations can be performed, for example, to obtain a derivative retaining the ability to induce protective immunity against S. aureus or to obtain a derivative that in addition to providing protective immunity also has a region that can achieve a particular purpose.

FIG. 2 provides a sequence comparison that includes full length sai-1 sequences (SEQ ID NOs: 7 and 8). The comparison illustrates amino acid differences between S. aureus isolates that can be used to guide the design of potential alterations to SEQ ID NO: 1 or 2. In addition, alterations can be made taking into account known properties of amino acids.

Generally, in substituting different amino acids to retain activity it is preferable to exchange amino acids having similar properties. Factors that can be taken into account for an amino acid substitution include amino acid size, charge, polarity, and hydrophobicity. The effect of different amino acid R-groups on amino acid properties are well known in the art. (See, for example, Ausubel, Current Protocols in Molecular Biology, John Wiley, 1987-2002, Appendix 1C.)

In exchanging amino acids to maintain activity, the replacement amino acid should have one or more similar properties such as approximately the same charge and/or size and/or polarity and/or hydrophobicity. For example, substituting valine for leucine, arginine for lysine, and asparagine for glutamine are good candidates for not causing a change in polypeptide functioning.

Alterations to achieve a particular purpose include those designed to facilitate production or efficacy of the polypeptide; or cloning of the encoded nucleic acid. Polypeptide production can be facilitated through the use of an initiation codon (e.g., coding for methionine) suitable for recombinant expression. The methionine may be later removed during cellular processing. Cloning can be facilitated by, for example, the introduction of restriction sites which can be accompanied by amino acid additions or changes.

Efficacy of a polypeptide to induce an immune response can be enhanced through epitope enhancement. Epitope enhancement can be performed using different techniques such as those involving alteration of anchor residues to improve peptide affinity for MHC molecules and those increasing affinity of the peptide-MHC complex for a T-cell receptor. (Berzofsky et al., Nature Review 1:209-219, 2001.)

Preferably, the polypeptide is a purified polypeptide. A “purified polypeptide” is present in an environment lacking one or more other polypeptides with which it is naturally associated and/or is represented by at least about 10% of the total protein present. In different embodiments, the purified polypeptide represents at least about 50%, at least about 75%, or at least about 95% of the total protein in a sample or preparation.

In an embodiment, the polypeptide is “substantially purified”. A substantially purified polypeptide is present in an environment lacking all, or most, other polypeptides with which the polypeptide is naturally associated. For example, a substantially purified S. aureus polypeptide is present in an environment lacking all, or most, other S. aureus polypeptides. An environment can be, for example, a sample or preparation.

Reference to “purified” or “substantially purified” does not require a polypeptide to undergo any purification and may include, for example, a chemically synthesized polypeptide that has not been purified.

Polypeptide stability can be enhanced by modifying the polypeptide carboxyl or amino terminus. Examples of possible modifications include amino terminus protecting groups such as acetyl, propyl, succinyl, benzyl, benzyloxycarbonyl or t-butyloxycarbonyl; and carboxyl terminus protecting groups such as amide, methylamide, and ethylamide.

Polypeptide purification can be enhanced by adding a group to the carboxyl or amino terminus to facilitate purification. Examples of groups that can be used to facilitate purification include polypeptides providing affinity tags. Examples of affinity tags include a six-histidine tag, trpE, glutathione and maltose-binding protein.

The ability of a polypeptide to produce an immune response can be enhanced using groups that generally enhance an immune response. Examples of groups that can be joined to a polypeptide to enhance an immune response against the polypeptide include cytokines such as IL-2. (Buchan et al., 2000. Molecular Immunology 37:545-552.)

Polypeptide Production

Polypeptides can be produced using standard techniques including those involving chemical synthesis and those involving purification from a cell producing the polypeptide. Techniques for chemical synthesis of polypeptides are well known in the art. (See e.g., Vincent, Peptide and Protein Drug Delivery, New York, N.Y., Decker, 1990.)

Techniques for polypeptide purification from a cell are illustrated in the Example provided below. Additional examples of purification techniques are well known in the art. (See for example, Ausubel, Current Protocols in Molecular Biology, John Wiley, 1987-2002.)

Obtaining polypeptides from a cell is facilitated using recombinant nucleic acid techniques to produce the polypeptide. Recombinant nucleic acid techniques for producing a polypeptide involve introducing, or producing, a recombinant gene encoding the polypeptide in a cell and expressing the polypeptide.

A recombinant gene contains nucleic acid encoding a polypeptide along with regulatory elements for polypeptide expression. The recombinant gene can be present in a cellular genome or can be part of an expression vector.

The regulatory elements that may be present as part of a recombinant gene include those naturally associated with the polypeptide encoding sequence and exogenous regulatory elements not naturally associated with the polypeptide encoding sequence. Exogenous regulatory elements such as an exogenous promoter can be useful for expressing a recombinant gene in a particular host or increasing the level of expression. Generally, the regulatory elements that are present in a recombinant gene include a transcriptional promoter, a ribosome binding site, a terminator, and an optionally present operator. A preferred element for processing in eukaryotic cells is a polyadenylation signal.

Expression of a recombinant gene in a cell is facilitated through the use of an expression vector. Preferably, an expression vector in addition to a recombinant gene also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Due to the degeneracy of the genetic code, a large number of different encoding nucleic acid sequences can be used to code for a particular polypeptide. The degeneracy of the genetic code arises because almost all amino acids are encoded by different combinations of nucleotide triplets or “codons”. Amino acids are encoded by codons as follows:
A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr-Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Suitable cells for recombinant nucleic acid expression of sai-1 related polypeptides are prokaryotes and eukaryotes. Examples of prokaryotic cells include E. coli; members of the Staphylococcus genus, such as S. aureus; members of the Lactobacillus genus, such as L. plantarum; members of the Lactococcus genus, such as L. lactis; and members of the Bacillus genus, such as B. subtilis. Examples of eukaryotic cells include mammalian cells; insect cells; yeast cells such as members of the Saccharomyces genus (e.g., S. cerevisiae), members of the Pichia genus (e.g., P. pastoris), members of the Hansenula genus (e.g., H. polymorpha), members of the Kluyveromyces genus (e.g., K. lactis or K. fragilis) and members of the Schizosaccharomyces genus (e.g., S. pombe).

Techniques for recombinant gene production, introduction into a cell, and recombinant gene expression are well known in the art. Examples of such techniques are provided in references such as Ausubel, Current Protocols in Molecular Biology, John Wiley, 1987-2002, and Sambrook et al., Molecular Cloning, A Laboratory Manual, 2^{nd} Edition, Cold Spring Harbor Laboratory Press, 1989.

If desired, expression in a particular host can be enhanced through codon optimization. Codon optimization includes use of more preferred codons. Techniques for codon optimization in different hosts are well known in the art.

Depending upon the host used for expression, sai-1 related polypeptides may contain post translational modifications. Reference to “polypeptide” or an “amino acid” sequence of a polypeptide includes polypeptides containing one or more amino acids having a structure of a post-translational modification from a host cell, such as a yeast host.

For example, in S. cerevisiae, the nature of the penultimate amino acid appears to determine whether the N-terminal methionine is removed. Furthermore, the nature of the penultimate amino acid also determines whether the N-terminal amino acid is N^{α}-acetylated (Huang et al., Biochemistry 26: (1987), 8242-8246, 1987). Thus, within the scope of this invention, the sai-1 related polypeptide may have an N^{α}-acetylated N-terminus and the N-terminal methionine may be removed, depending on which amino acid is in the penultimate position.

In addition, if the sai-1 related polypeptide is targeted for secretion due to the presence of a secretory leader (e.g., signal peptide), the protein may be modified by N-linked or O-linked glycosylation. (Kukuruzinska et al., Ann. Rev. Biochem. 56:915-944, 1987.)

Adjuvants

Adjuvants are substances that can assist an immunogen in producing an immune response. Adjuvants can function by different mechanisms such as one or more of the following: increasing the antigen biologic or immunologic half-life; improving antigen delivery to antigen-presenting cells; improving antigen processing and presentation by antigen-presenting cells; and inducing production of immunomodulatory cytokines. (Vogel, Clinical Infectious Diseases 30(suppl. 3):S266-270, 2000.)

A variety of different types of adjuvants can be employed to assist in the production of an immune response. Examples of particular adjuvants include aluminum hydroxide, aluminum phosphate, or other salts of aluminum, calcium phosphate, DNA CpG motifs, monophosphoryl lipid A, cholera toxin, E. coli heat-labile toxin, pertussis toxin, muramyl dipeptide, Freund's incomplete adjuvant, MF59, SAF, immunostimulatory complexes, liposomes, biodegradable microspheres, saponins, nonionic block copolymers, muramyl peptide analogues, polyphosphazene, synthetic polynucleotides, IFN-γ, IL-2 and IL-12. (Vogel Clinical Infectious Diseases 30(suppl 3):S266-270, 2000, Klein et al., Journal of Pharmaceutical Sciences 89:311-321, 2000.)

Patients For Inducing Protective Immunity

A “patient” refers to a mammal capable of being infected with S. aureus. A patient can be treated prophylactically or therapeutically. Prophylactic treatment provides sufficient protective immunity to reduce the likelihood, or severity, of a S. aureus infection. Therapeutic treatment can be performed to reduce the severity of a S. aureus infection.

Prophylactic treatment can be performed using a vaccine containing an immunogen described herein. Such treatment is preferably performed on a human. Vaccines can be administered to the general population or to those persons at an increased risk of S. aureus infection.

Persons with an increased risk of S. aureus infection include health care workers; hospital patients; patients with a weakened immune system; patients undergoing surgery; patients receiving foreign body implants, such a catheter or a vascular device; patients facing therapy leading to a weakened immunity; and persons in professions having an increased risk of burn or wound injury. (The Staphylococci in Human Disease, Crossley and Archer (ed.), Churchill Livingstone Inc. 1997.)

Non-human patients that can be infected with S. aureus include cows, pigs, sheep, goats, rabbits, horses, dogs, cats and mice. Treatment of non-human patients is useful in protecting pets and livestock, and in evaluating the efficacy of a particular treatment.

Combination Vaccines

SEQ ID NO: 1 related polypeptides can be used alone, or in combination with other immunogens, to induce an immune response. Additional immunogens that may be present include: one or more additional S. aureus immunogens, such as those referenced in the Background of the Invention supra; one or more immunogens targeting one or more other Staphylococcus organisms such as S. epidermidis, S. haemolyticus, S. warneri, or S. lugunensis; and one or more immunogens targeting other infections organisms.

Animal Model System

An animal model system was used to evaluate the efficacy of an immunogen to produce a protective immune response against Staphylococcus. Two obstacles encountered in setting up a protective animal model were: (1) very high challenge dose needed to overcome innate immunity and (2) death rate too fast to detect a protective response. Specifically, after bacterial challenge mice succumbed to infection within 24 hours which did not provide sufficient time for the specific immune responses to resolve the infection. If the dose was lowered both control and immunized mice survived the infection.

These obstacles were addressed by developing a slow kinetics lethality model involving S. aureus prepared from cells in stationary phase, appropriately titrated, and intravenously administered. This slow kinetics of death provides sufficient time for the specific immune defense to fight off the bacterial infection (e.g., 10 days rather 24 hours).

S. aureus cells in stationary phase can be obtained from cells grown on solid medium. They can also be obtained from liquid, however the results with cells grown on solid media were more reproducible. Cells can conveniently be grown overnight on solid medium. For example, S. aureus can be grown from about 18 to about 24 hours under conditions where the doubling time is about 20-30 minutes.

S. aureus can be isolated from solid or liquid medium using standard techniques to maintain Staphylococcus potency. Isolated Staphylococcus can be stored, for example, at −70° C. as a washed high density suspension (>10⁹ colony forming units (CFU)/mL) in phosphate buffered saline containing glycerol.

The S. aureus challenge should have a potency providing about 80 to 90% death in an animal model over a period of about 7 to 10 days starting on the first or second day. Titration experiments can be performed using animal models to monitor the potency of the stored Staphylococcus inoculum. The titration experiments can be performed about one to two weeks prior to an inoculation experiment.

Initial potency for titration experiments can be based on previous experiments. For S. aureus and the animal model strain Becker a suitable potency was generally found in the range of 5×10⁸ to 8×10⁸ CFU/ml.

Administration

Immunogens can be formulated and administered to a patient using the guidance provided herein along with techniques well known in the art. Guidelines for pharmaceutical administration in general are provided in, for example, Vaccines Eds. Plotkin and Orenstein, W.B. Sanders Company, 1999; Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990, each of which are hereby incorporated by reference herein.

Pharmaceutically acceptable carriers facilitate storage and administration of an immunogen to a patient. Pharmaceutically acceptable carriers may contain different components such as a buffer, sterile water for injection, normal saline or phosphate buffered saline, sucrose, histidine, salts and polysorbate.

Immunogens can be administered by different routes such as subcutaneous, intramuscular, or mucosal. Subcutaneous and intramuscular administration can be performed using, for example, needles or jet-injectors.

Suitable dosing regimens are preferably determined taking into account factors well known in the art including age, weight, sex and medical condition of the patient; the route of administration; the desired effect; and the particular compound employed. The immunogen can be used in multi-dose vaccine formats. It is expected that a dose would consist of the range of 1.0 μg to 1.0 mg total polypeptide, in different embodiments of the present invention the range is 0.01 mg to 1.0 mg and 0.1 mg to 1.0 mg.

The timing of doses depends upon factors well known in the art. After the initial administration one or more booster doses may subsequently be administered to maintain or boost antibody titers. An example of a dosing regime would be day 1, 1 month, a third dose at either 4, 6 or 12 months, and additional booster doses at distant times as needed.

Generation of Antibodies

A SEQ ID NO: 1 related polypeptide can be used to generate antibodies and antibody fragments that bind to the polypeptide or to S. aureus. Such antibodies and antibody fragments have different uses including use in polypeptide purification, S. aureus identification, or in therapeutic or prophylactic treatment against S. aureus infection.

Antibodies can be polyclonal or monoclonal. Techniques for producing and using antibodies are well known in the art. Examples of such techniques are described in Ausubel, Current Protocols in Molecular Biology, John Wiley, 1987-2002, Harlow et al., Antibodies, A Laboratory Manual, Cold Spring Harbor Laboratory, 1988, and Kohler et al., Nature 256:495-497, 1975.

### EXAMPLES

Examples are provided below further illustrating different features of the present invention. The examples also illustrate useful methodology for practicing the invention. These examples do not limit the claimed invention.

### Example 1

### Use of SEQ ID NO: 3 to Provide Protective Immunity

This example illustrates the ability of SEQ ID NO: 1 related polypeptides to provide protective immunity in a model. SEQ ID NO: 3, a His-tagged derivative of SEQ ID NO: 1, was used to provide protective immunity.

Sai-1 Cloning and Expression

A sai-1 DNA sequence was translated using Vector NTI software and the resulting 355 amino acid sequence was analyzed. PCR primers were designed to amplify the gene starting at the first asparagine residue and ending prior to the stop codon at the terminal asparagine residue. These PCR primers also had additional NcoI (forward primer) and XhoI (reverse primer) sites to facilitate cloning into the expression vector.

The protein was designed to be expressed from the pET28 vector with the terminal His residues and the stop codon encoded by the vector. In addition, a glycine residue was added to the protein after the methionine initiator for Sai-N. Sai-C has a carboxyl terminal His-tag and is composed of the 350 amino acid sai-1 protein and an additional 8 amino acids at the carboxyl end comprising the poly His tail and vector sequences. Sai-N had an amino terminal His-tag and is composed of a 351 amino acid sai-1 protein (includes the glycine insert), with an additional 46 amino acids comprising the poly His tail and vector sequences.

PCR amplified sequences were ligated into the pET28 vector (Novagen) using the NcoI/XhoI sites that had been engineered into the PCR primers and introduced into E. coli DH5α (Invitrogen) by heat shock. PCR amplified sequences were ligated into the pET28 vector (Novagen) using the NcoI/XhoI sites that had been engineered into the PCR primers and introduced into E. coli DH5α (Invitrogen) by heat shock. Colonies were selected, grown in LB with 30 μg/mL kanamycin, DNA minipreps made (Promega), and insert integrity determined by restriction digestion and PCR. Four minipreps with correct insert size were sequenced using the primers listed in Table 1. A clone was selected containing no DNA changes from the desired sequence.

TABLE 1

| SEQ ID NO: | Description | Sequence |
|---|---|---|
| 12 | Sai1-CF | GAGATATACCATGGGCACAAAACATTATTTAAACAGT |
| 13 | Sai1-CR | CCGGCGGCCCTCGAGTTTAGATTCTTTTCTTTTGAA |
| 14 | Sai1-NF | GAGATATACCATGGGCACAAAACATTATTTAAACAGT |
| 15 | Sai1-NR | CCGGCGGCCCTCGAGTTATTTAGATTCTTTTCTTTTGAA |
| 16 | Sai1-C2F | GAGATATACCATGGGCACACAAGTTTCTCAAGCAACATCAC |
| 17 | Sai1-C2R | GGTGGTGCTCGAGAGTTTTTGGTAATTCTTTAGCTT |
| 18 | Sai1-N2F | GAGATATCATATGGGCACACAAGTTTCTCAAGCAACATCAC |
| 19 | Sai1-N2R | GGTGGTGCTCGAGTCAAGTTTTTGGTAATTCTTTAGCTT |

E. coli HMS174(DE3) cells (Novagen) were transformed and grown on LB plates containing kanamycin (30 ug/ml); 3 colonies were selected for expression testing. Liquid LB (kanamycin) cultures were incubated at 37° C., 250 rpm until the A₆₀₀ was between 0.6 and 1.0 and then induced by the addition of IPTG to final concentrations of 1 mM followed by three hours further incubation. Cultures were harvested by centrifugation at 5000×g for 5 minutes at 4° C. Cells were resuspended in 500 μl lysis buffer (Bug Buster, with protease inhibitors, Novagen). An equal volume of loading buffer (supplemented with β-mecapto ethanol to 5% final volume) was added prior to heating the samples at 70° C. for 5 minutes. Extracts were run on Novex 4-20% Tris-Glycine gels and assayed for protein (Coomassie Blue stained) and blotted onto nitrocellulose and probed with anti-HIS6 antibodies (Zymedd). The expression observed was extremely low.

The protein was re-analyzed; a putative signal sequence was removed as was the down stream region from the LPXTG motif. These PCR primers also had additional NdeI (forward primer) and XhoI (reverse primer) sites to facilitate cloning into the expression vector.

The protein was designed to be expressed from the pET28 vector with the terminal. His residues and the stop codon encoded by the vector. In addition, a glycine residue was added to the protein after the methionine initiator. Sai-N2 (SEQ ID NO: 3) contains an amino His-tag. Sai-C2 (SEQ ID NO: 5) contains a carboxyl terminal His-tag.

PCR amplified sequences were ligated into the pET28 vector (Novagen) using the NdeI/XhoI sites that had been engineered into the PCR primers and introduced into E. coli DH5α (Invitrogen) by heat shock. PCR amplified sequences were ligated into the pET28 vector (Novagen) using the NdeI/XhoI sites that had been engineered into the PCR primers and introduced into E. coli DH5α (Invitrogen) by heat shock. Colonies were selected, grown in LB with 30 μg/mL kanamycin, DNA minipreps made (Promega), and insert integrity determined by restriction digestion and PCR. Four minipreps with correct insert size were sequenced using the primers listed in Table 1. A clone was selected containing no DNA changes from the desired sequence.

E. coli HMS174(DE3) cells (Novagen) were transformed and grown on LB plates containing kanamycin (30 ug/ml); 3 colonies were selected for expression testing. Liquid LB (kanamycin) cultures were incubated at 37° C., 250 rpm until the A₆₀₀ was between 0.6 and 1.0 and then induced by the addition of IPTG to final concentrations of 1 mM followed by three hours further incubation. Cultures were harvested by centrifugation at 5000×g for 5 minutes at 4° C. Cells were resuspended in 500 μl lysis buffer (Bug Buster, with protease inhibitors, Novagen). An equal volume of loading buffer (supplemented with β-mecapto ethanol to 5% final volume) was added prior to heating the samples at 70° C. for 5 minutes. Extracts were run on Novex 4-20% Tris-Glycine gels and assayed for protein (Coomassie Blue stained) and blotted onto nitrocellulose and probed with anti-HIS6 antibodies (Zymedd).

SEQ ID NO: 3 Purification

Recombinant E. coli cells (46 grams wet cell weight) were suspended in Lysis Buffer (50 mM sodium phosphate, pH 8.0, 0.15 M NaCl, 2 mM MgCl₂, 10 mM imidazole, 0.1% Tween™-80, and 0.02% sodium azide) at 3 ml per gram of cell wet weight. Protease Inhibitor Cocktail for use with poly-(Histidine)-tagged proteins (Roche #1873580) was added to the suspension at 1 tablet per 15 grams of cell paste. Benzonase™ (EM Ind.) was added to 1 μL/mL. Cell lysis was accomplished by passing the suspension through a microfluidizer at 14,000 PSI (Microfluidics Model 110S) three times. The cell suspension was cooled on ice between each pass so that the temperature remained below 25° C. Cell debris was pelleted at 11,000×g for 30 minutes at 4° C., and the supernatant retained.

Proteins bearing a His-tag were purified from the supernatant. The supernatant was mixed with 12 mL of Ni⁺-NTA agarose (Qiagen) at 4° C. with gentle inversion for 18 hours. The mixture was poured into an open column (1.5 cm×20 cm) and the non-bound fraction was collected in bulk. The column was washed with Wash Buffer (50 mM sodium phosphate, pH 8.0, 0.3 M NaCl, 20 mM imidazole, and 0.1% Tween™-80). His-tagged protein was eluted with a step gradient of 300 mM imidazole, 20 mM Tris-HCl, pH 8, 0.3 M NaCl, 0.1% Tween™-80.

Fractions containing SEQ ID NO: 3 polypeptide were detected by Coomassie stained SDS-PAGE and pooled. Pooled fractions were filtered through a 0.2 micron filter to remove particulate material, and were applied on a size-exclusion column (Sephacryl S-300 26/60 column, Amersham Biosciences) and eluted at 1 mL/min with 30 MM MOPS pH 7.0, 0.3 M NaCl, and 10% glycerol. Fractions containing SEQ ID NO: 3 polypeptide were detected by Coomassie stained SDS-PAGE and Western blotting (anti-tetra His Mab, Qiagen). Protein was determined by BCA (Pierce). Purity was determined by densitometry of Coomassie stained gels.

Preparation of S. aureus Challenge

S. aureus was grown on TSA plates at 37° C. overnight. The bacteria were washed from the TSA plates by adding 5 ml of PBS onto a plate and gently resuspending the bacteria with a sterile spreader. The bacterial suspension was spun at 6000 rpm for 20 minutes using a Sorvall RC-5B centrifuge (DuPont Instruments). The pellet was resuspended in 16% glycerol and aliquots were stored frozen at −70° C.

Prior to use, inocula were thawed, appropriately diluted and used for infection. Each stock was titrated at least 3 times to determine the appropriate dose inducing slow kinetics of death in naive mice. The potency of the bacterial inoculum (80 to 90% lethality) was constantly monitored to assure reproducibility of the model. Ten days before each challenge experiment, a group of 10 control animals (immunized with adjuvant alone) were challenged and monitored.

Protection Studies for a SEQ ID NO: 3 Polypeptide

Twenty BALB/c mice were immunized with three doses of a SEQ ID NO: 3 polypeptide (20 μg per dose) on aluminum hydroxyphosphate adjuvant (450 μg per dose). Aluminum hydroxyphosphate adjuvant (AHP) is described by Klein et al., Journal of Pharmaceutical Sciences 89, 311-321, 2000. The doses were administered as two 50 μl injections on days 0, 7 and 21. The mice were bled on day 28, and their sera were screened by ELSIA for reactivity to the SEQ ID NO: 3 polypeptide.

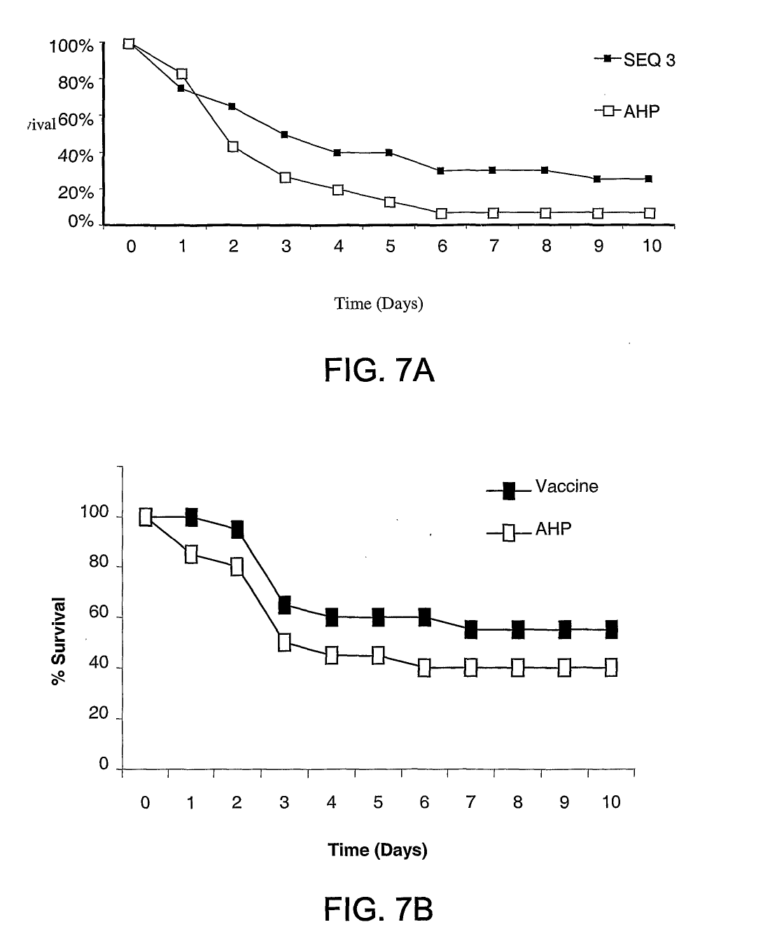

On day 35 of the experiment the mice were challenged by intravenous injection of S. aureus grown at a dose (8.0×10⁸ CFU ml). The mice were monitored over a 10 day period for survival. At the end of the experiment 5 mice survived the SEQ ID NO: 3 polypeptide immunized group, compared to 2 surviving in the AHP control group containing 30 mice. The experiment was repeated using 20 immunized mice and 20 control mice. Results for both experiments are shown in FIGS. 7A and 7B.

### Example 2

### Intracellular Expression from Nucleic Acid Encoding SEQ ID NO: 1 Related Proteins

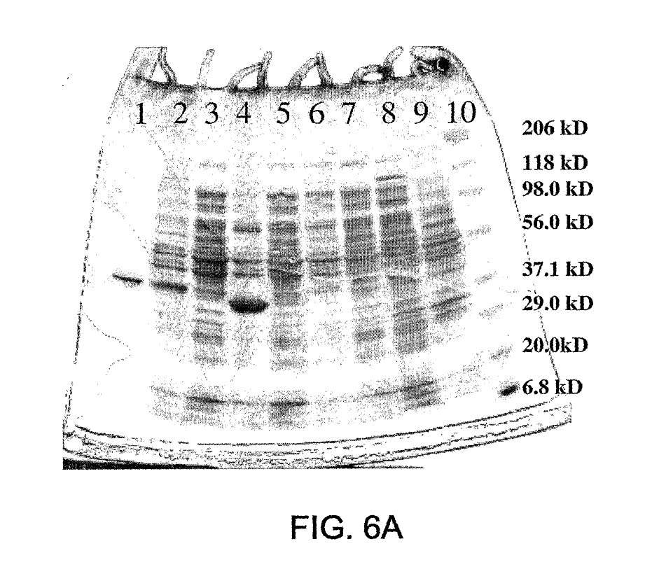

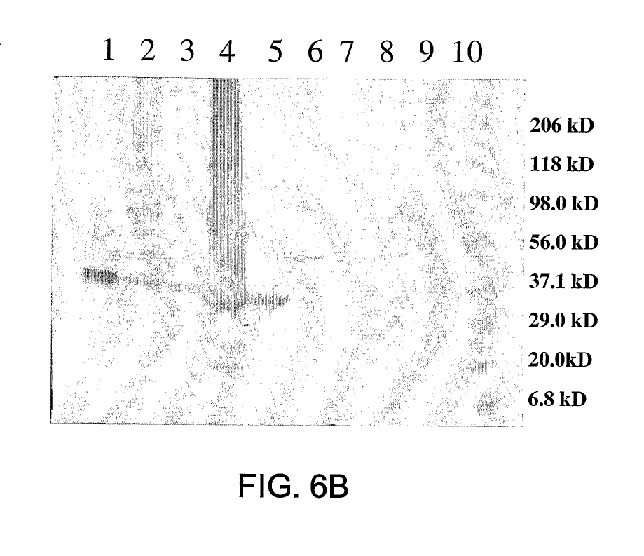

FIGS. 6A and 6B show an exemplary Coomassie stain of an SDS-PAGE gel and a Western blot, respectively, comparing intracellular expression from nucleic acid encoding SEQ ID NO: 1 related proteins. The Western blot was probed using an anti-his antibody. Lanes-1, Purified SEQ ID NO: 3 (100 ng); 2, SEQ ID NO: 3 E. coli crude lysate (with induction); 3, SEQ ID NO: 3 E. coli crude lysate (no induction); 4, SEQ ID NO: 5 E. coli crude lysate (with induction); 5, SEQ ID NO: 5 E. coli crude lysate (no induction); 6, SEQ ID NO: 6 E. coli crude lysate (with induction); 7, SEQ ID NO: 6 E. coli crude lysate (no induction); 8, SEQ ID NO: 9 E. coli crude lysate (with induction); 9, SEQ ID NO: 9 E. coli crude lysate (no induction); 10, Standard.

Other embodiments are within the following claims. While several embodiments have been shown and described, various modifications may be made without departing from the spirit and scope of the present invention.

1. An polypeptide immunogen comprising an amino acid sequence at least 85% identical to SEQ ID NO: 1, wherein said polypeptide provides protective immunity against S. aureus and wherein if one or more additional polypeptide regions are present said additional regions do not provide a carboxyl terminus containing amino acids 261-294 of SEQ ID NO: 7.

2. The polypeptide of claim 1, wherein said polypeptide consists of an amino acid sequence at least 94% identical to either SEQ ID NO: 1 or SEQ ID NO: 2.

3. The polypeptide of claim 1, wherein said polypeptide consists essentially of amino acids 3-260 of SEQ ID NO: 1 or 3-264 of SEQ ID NO: 2.

4. The polypeptide of claim 3, wherein said polypeptide consists of an amino acid sequence of SEQ ID NO: 1.

5. An immunogen comprising the polypeptide of claim 1, wherein said immunogen consists of said polypeptide and one or more additional regions moieties covalently joined to said polypeptide at the carboxyl terminus or amino terminus, wherein each region or moiety is independently selected from a region or moiety having at least one of the following properties: enhances the immune response, facilitates purification, or facilitates polypeptide stability.

6. A composition able to induce a protective immune response in a patient comprising an immunologically effective amount of the immunogen of claim 5 and a pharmaceutically acceptable carrier.

7. The composition of claim 6, wherein said composition further comprises an adjuvant.

8. A nucleic acid comprising a recombinant gene comprising a nucleotide sequence encoding the polypeptide of claim 1.

9. The nucleic acid of claim 8, wherein said nucleic acid is an expression vector.

10. A recombinant cell comprising a recombinant gene comprising a nucleotide sequence encoding the polypeptide of claim 1.

11. A method of making a S. aureus polypeptide that provides protective immunity comprising the steps of:
(a) growing the recombinant cell of claim 10 under conditions wherein a polypeptide is expressed; and
(b) purifying said polypeptide.

12. A method of inducing a protective immune response in a patient comprising the step of administering to said patient an immunologically effective amount of a polypeptide immunogen comprising an amino acid at least 85% identical to SEQ ID NO: 1.

13. The method of claim 12, wherein said patient is a human.

14. The method of claim 13, wherein said patient is treated prophylactically against S. aureus infection.

15. The method of claim 12, wherein said immunogen is the immunogen of claim 1.


SEQUENCE LISTING (19 sequences)

SEQ ID NO: 1 - protein, 260 aa, Artificial Sequence
  truncated derivative of sai-1
MGTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKT

SEQ ID NO: 2 - protein, 264 aa, S. aureus
MGTQVSQATSQPINFQVQKDGSSEKSHMDDYMQHPGKVIKQNNKYYFQAVLNNASFWKEYKFYNANNQELATTVVNDDKKADTRTINVAVEPGYKSLTTKVHIVVPQINYNHRYTTHLEFEKAIPTLADAAKPNNVKPVQPKPAQPKTPTEQTKPVQPKVEKVKPAVTAPSKNENRQTTKVVSSEATKDQSQTQSARTVKTTQTAQDQNKVQTPVKDVATAKSESNNQAVSDNKSQQTNKVTKQNEVHKQGPSKDSKAKELPKT

SEQ ID NO: 3 - protein, 280 aa, Artificial Sequence
  amino His-tagged construct of SEQ ID NO 1
MGSSHHHHHHSSGLVPRGSHMGTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKT

SEQ ID NO: 4 - protein, 284 aa, Artificial Sequence
  amino His-tagged construct of SEQ ID NO 2
MGSSHHHHHHSSGLVPRGSHMGTQVSQATSQPINFQVQKDGSSEKSHMDDYMQHPGKVIKQNNKYYFQAVLNNASFWKEYKFYNANNQELATTVVNDDKKADTRTINVAVEPGYKSLTTKVHIVVPQINYNHRYTTHLEFEKAIPTLADAAKPNNVKPVQPKPAQPKTPTEQTKPVQPKVEKVKPAVTAPSKNENRQTTKVVSSEATKDQSQTQSARTVKTTQTAQDQNKVQTPVKDVATAKSESNNQAVSDNKSQQTNKVTKQNEVHKQGPSKDSKAKELPKT

SEQ ID NO: 5 - protein, 268 aa, Artificial Sequence
  carboxyl His-tagged construct of SEQ ID NO 1
MGTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTLEHHHHHH

SEQ ID NO: 6 - protein, 395 aa, Artificial Sequence
  amino His-tagged construct of SEQ ID NO 7
MHHHHHHSSGLVPRGSGMKETAAAKFERQHMDSPDLGTDDDDKAMGTKHYLNSKYQSEQRSSAMKKITMGTASIILGSLVYIGADSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 7 - protein, 350 aa, S. aureus
MTKHYLNSKYQSEQRSSAMKKITMGTASIILGSLVYIGADSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 8 - protein, 354 aa, S. aureus
MTKHYLNSKYQSEQRSSAMKKITMGTASIILGSLVYIGADSQQVNAATEATNATNNQSTQVSQATSQPINFQVQKDGSSEKSHMDDYMQHPGKVIKQNNKYYFQAVLNNASFWKEYKFYNANNQELATTVVNDDKKADTRTINVAVEPGYKSLTTKVHIVVPQINYNHRYTTHLEFEKAIPTLADAAKPNNVKPVQPKPAQPKTPTEQTKPVQPKVEKVKPAVTAPSKNENRQTTKVVSSEATKDQSQTQSARTVKTTQTAQDQNKVQTPVKDVATAKSESNNQAVSDNKSQQTNKVTKQNEVHKQGPSKDSKAKELPKTGLTSVDNFISTVAFATLALLGSLSLLLFKRKESK

SEQ ID NO: 9 - protein, 358 aa, Artificial Sequence
  carboxyl His-tagged construct of SEQ ID NO 7
MTKHYLNSKYQSEQRSSAMKKITMGTASIILGSLVYIGADSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESKLEHHHHHH

SEQ ID NO: 10 - DNA, 843 bp, Artificial Sequence
  nucleic acid sequence encoding SEQ ID NO 3
atgggcagcagccatcatcatcatcatcacagcagcggcctggtgccgcgcggcagccatatgggcacacaagtttctcaagcaacatcacaaccaattaatttccaagtgcaaaaagatggctcttcagagaagtcacacatggatgactatatgcaacaccctggtaaagtaattaaacaaaataataaatattatttccaaaccgtgttaaacaatgcatcattctggaaagaatacaaattttacaatgcaaacaatcaagaattagcaacaactgttgttaacgataataaaaaagcggatactagaacaatcaatgttgcagttgaacctggatataagagcttaactactaaagtacatattgtcgtgccacaaattaattacaatcatagatatactacgcatttggaatttgaaaaagcaattcctacattagctgacgcagcaaaaccaaacaatgttaaaccggttcaaccaaaaccagctcaacctaaaacacctactgagcaaactaaaccagttcaacctaaagttgaaaaagttaaacctactgtaactacaacaagcaaagttgaagacaatcactctactaaagttgtaagtactgacacaacaaaagatcaaactaaaacacaaactgctcatacagttaaaacagcacaaactgctcaagaacaaaataaagttcaaacacctgttaaagatgttgcaacagcgaaatctgaaagcaacaatcaagctgtaagtgataataaatcacaacaaactaacaaagttacaaaacataacgaaacgcctaaacaagcatctaaagctaaagaattaccaaaaacttga

SEQ ID NO: 11 - DNA, 855 bp, Artificial Sequence
  nucleic acid sequence encoding SEQ ID NO 4
atgggcagcagccatcatcatcatcatcacagcagcggcctggtgccgcgcggcagccatatgggcacacaagtttctcaagcaacatcacaaccaattaatttccaagtgcaaaaagatggctcttcagagaagtcacacatggatgactatatgcaacaccctggtaaagtgattaaacaaaataataaatattatttccaagctgtattgaacaacgcatcattctggaaagaatacaaattttacaatgcaaacaatcaagaattagcaacaactgttgttaacgatgataaaaaagctgacactagaacaatcaatgttgctgttgaacctgggtataagagtttaactacaaaagtacatattgtcgtgccacaaattaattataatcatagatatactacgcatttagaatttgaaaaagcaattcctacattagctgacgcagcaaaaccaaacaatgttaaaccggttcaaccaaaacctgctcaacctaaaacacctactgagcaaacgaaaccagttcaacctaaagttgaaaaagttaaacctgctgtaactgcaccaagcaaaaatgaaaacagacaaactacaaaagttgtaagtagtgaagctacaaaagatcaaagtcaaacacaaagtgctcgtacagtgaaaacaacacaaacagctcaagatcaaaataaagttcaaacacctgttaaagatgttgcaacagcgaaatctgaaagcaacaatcaagctgtaagtgacaataaatcacaacaaactaacaaagttacaaaacaaaacgaagttcataaacaaggaccttcaaaagattctaaagctaaagaattaccaaaaacttga

SEQ ID NO: 12 - DNA, 37 bp, Artificial Sequence
  primer
gagatataccatgggcacaaaacattatttaaacagt

SEQ ID NO: 13 - DNA, 36 bp, Artificial Sequence
  primer
ccggcggccctcgagtttagattcttttcttttgaa

SEQ ID NO: 14 - DNA, 37 bp, Artificial Sequence; identical to SEQ ID NO: 12
  primer

SEQ ID NO: 15 - DNA, 39 bp, Artificial Sequence
  primer
ccggcggccctcgagttatttagattcttttcttttgaa

SEQ ID NO: 16 - DNA, 41 bp, Artificial Sequence
  primer
gagatataccatgggcacacaagtttctcaagcaacatcac

SEQ ID NO: 17 - DNA, 36 bp, Artificial Sequence
  primer
ggtggtgctcgagagtttttggtaattctttagctt

SEQ ID NO: 18 - DNA, 41 bp, Artificial Sequence
  primer
gagatatcatatgggcacacaagtttctcaagcaacatcac

SEQ ID NO: 19 - DNA, 39 bp, Artificial Sequence
  primer
ggtggtgctcgagtcaagtttttggtaattctttagctt